(19) United States
(12) Patent Application Publication
(10) Pub. No.: US 2026/0096738 A1
(43) Pub. Date: Apr. 9, 2026

(54) DETERMINING A CONCENTRATION OF GLUCOSE IN A SUBJECT'S BLOOD

(71) Applicant: Afon Technology Limited, Caldicot (GB)

(72) Inventors: Mohamed Sabih CHAUDHRY, Caldicot (GB); Muhammed QURESHI, Caldicot (GB); Bradley LOVE, Caldicot (GB); Hassen KAYALI, Caldicot (GB)

(21) Appl. No.: 19/416,470

(22) Filed: Dec. 11, 2025

Related U.S. Application Data
(63) Continuation of application No. PCT/EP2024/067344, filed on Jun. 20, 2024.

(30) Foreign Application Priority Data
Jun. 23, 2023 (GB) 2309524.3

(51) Int. Cl. A61B 5/0507; A61B 5/00; A61B 5/01; A61B 5/145

(52) U.S. Cl. CPC A61B 5/0507; A61B 5/01; A61B 5/14532; A61B 5/4875; A61B 5/681; A61B 5/7267; A61B 5/746; A61B 2560/0242; A61B 2560/0462; A61B 2562/0219; A61B 2562/0271; A61B 2562/029

(57) ABSTRACT

According to an aspect, there is provided a computer-implemented method (200) of determining a concentration of glucose in a subject's blood, the method comprising: acquiring (202) first measurement data indicative of a response resulting from an electromagnetic (EM) signal interacting with the subject's blood in a body part of the subject; acquiring (204) second measurement data indicative of at least one parameter associated with the subject; and using (206) a predictive model to infer a concentration of glucose in the subject's blood from the first measurement data and the second measurement data, the predictive model having been trained to infer a concentration of glucose in the subject's blood from the first measurement data and the second measurement data.

### CROSS REFERENCE TO RELATED APPLICATIONS

This application is a continuation of International Application No. PCT/EP2024/067344, filed Jun. 20, 2024, which claims the benefit of GB Application No. 2309524.3, filed Jun. 23, 2023, each of which are herein incorporated by reference in their entirety.

### FIELD OF THE INVENTION

The invention relates to determining a concentration of glucose in a subject's blood and, more specifically, to a method and an apparatus for determining a concentration of glucose in a subject's blood.

### BACKGROUND OF THE INVENTION

Diabetes mellitus, more commonly known as diabetes, is a disease in which the body does not produce enough insulin, or the insulin it produces is ineffective. Insulin is a hormone produced by the pancreas that the body uses to convert sugar and starches into energy. In other words, insulin is the hormone that unblocks cells of the body, allowing glucose to enter these cells to provide food to keep them alive.

People suffering from diabetes are known as diabetics, and diabetics suffer because as glucose (blood sugar) cannot enter their body's cells normally, glucose concentrations in the body (and in particular in the blood) build-up. Without appropriate and timely intervention or treatment, the cells within the body can end up being starved and blood sugar levels can remain high, leading to various health problems over time.

Accordingly, it is generally recommended that diabetics monitor their blood sugar levels regularly, to ensure that, in the event that the level increases or decreases beyond safe limits, appropriate and timely action can be taken.

Existing blood-glucose monitoring techniques fall generally into two categories: invasive and non-invasive. The most common invasive blood-glucose monitoring technique involves the withdrawal of blood from the patient, for example by pricking a finger or other body part to withdraw blood, then depositing one or more drops of the blood onto a reagent carrier strip having a glucose testing substance thereon. The testing substances change colour or shading in response to the detected amount of blood-glucose. A colour chart is then used to determine the associated numerical value of blood-glucose. One of the technical short falls of this technique is that measurement sensitivity is somewhat limited due to the finite range of colours and boundary spacing.

The pricking procedure itself can be painful, particularly for children, and it is often necessary to repeat the procedure several times throughout the day, which can be inconvenient and time consuming. Forgetting to perform the procedure, and failing to check the blood-glucose level can lead to complications, especially if an occurrence of a particularly high or low blood-glucose level is missed.

An alternative to performing repeated blood withdrawal procedures is to use an implanted device, which requires a complex medical procedure to be performed to insert a measurement instrument inside the patient's body. The medical procedure can require a stay in hospital, and can be uncomfortable and painful for the patient.

A further alternative invasive monitoring technique, which enable continuous glucose monitoring, involves the patient wearing a device mounted to a body part (e.g., their arm). The device may take the form of a patch, which has a small needle that penetrates the wearer's skin, and a sensor that takes real-time measurements of the blood glucose level. Again, such a device can be uncomfortable or painful for a patient and, due to its invasive nature, comes with the risk of infection.

Non-invasive alternatives have been proposed to mitigate some of the issues mentioned above. For example, a patient may wear a wearable device, which performs continuous or periodic measurements of the patient's blood glucose level. Some such devices transmit radiofrequency (RF) signals into a body part of the patient, and measure a response of the RF signal resulting from the RF signal's interaction with blood in the body part of the patient. However, many factors affect the response signal, such as how tight the device is worn on the body part, the anatomical dimensions of the body part, and the extent to which the patient is moving while the measurement takes place. While some of these factors can, to some extent, be accounted for when determining the blood glucose concentration, it can be difficult to determine an accurate measurement consistently.

There is therefore a desire for a non-invasive technique for determining a concentration of glucose in a person's blood, which helps to mitigate at least one of the above-identified problems.

### SUMMARY OF THE INVENTION

The present disclosure provides a technique for determining a concentration of glucose in a person's blood which at least partially addresses some of the problems discussed above. The technique disclosed herein is considered non-invasive, and is capable of providing an accurate indication of the glucose concentration by measuring a response from an electromagnetic (EM) signal interacting with blood in a body part of a subject and measuring a parameter associated with the subject, then providing the measurements to a predictive model in order to determine the glucose concentration. The inventors have recognised that with just these measurements, a predictive model that has been trained to determine or infer a glucose concentration based on those measurements is capable of providing an accurate indication which can be used to monitor the subject's blood glucose levels.

According to a first specific aspect, there is provided a computer-implemented method of determining a concentration of glucose in a subject's blood, the method comprising: acquiring first measurement data indicative of a response resulting from an electromagnetic (EM) signal interacting with the subject's blood in a body part of the subject: acquiring second measurement data indicative of at least one parameter associated with the subject; and using a predictive model to infer a concentration of glucose in the subject's blood from the first measurement data and the second measurement data, the predictive model having been trained to infer a concentration of glucose in the subject's blood from the first measurement data and the second measurement data.

By training a predictive model in this way, concentrations of glucose in blood of subjects can be determined using just the first measurement data and the second measurement data, which can be obtained in a quick, painless, non-invasive manner. By taking into account the data indicative of at least one parameter associated with the subject, it may be possible for the predictive model to infer a blood glucose concentration that is far more accurate than if the first measurement data alone is used to determine a blood glucose concentration.

In some embodiments, the method may further comprise, responsive to determining that the inferred concentration of glucose in the subject's blood meets a threshold condition, generating an alert signal for delivery to a recipient device.

The first measurement data may comprise data indicative of a frequency corresponding to an absolute minimum in the response resulting from the EM signal interacting with the subject's blood in the body part of the subject.

In some embodiments, the EM signal may have a frequency in the range 200 kHz to 10 GHz.

The first measurement data may, in some embodiments, comprise: a) data indicative of a response resulting from an EM signal in a first EM waveband interacting with the subject's blood in the body part of the subject; and b) data indicative of a response resulting from an EM signal in a second EM waveband interacting with the subject's blood in the body part of the subject.

The EM signal in the first waveband may have a frequency of between 4.8 GHz and 5.4 GHz, and the EM signal in the second waveband may have a frequency of between 6.3 GHz and 6.7 GHz.

In some embodiments, the first measurement data may comprise data indicative of a response resulting from the EM signal reflecting from the subject's blood as the EM signal interacts with the body part of the subject.

The at least one parameter associated with the subject may comprise a parameter selected from a group comprising: a temperature of a surface of skin of the body part: an ambient temperature around the body part: a level of moisture on a surface of skin of the body part: an ambient humidity around the body part; and a level of motion of the body part.

In some embodiments, the method may further comprise processing the first measurement data by: plotting an amplitude of the response as a function of frequency: determining an approximate location of an absolute minimum in the plot: smoothing data forming the absolute minimum; and determining, based on the smoothed data, an estimation of a frequency corresponding to the amplitude at the absolute minimum.

The method may, in some embodiments, further comprise, prior to acquiring the first measurement data, controlling a signal generator to generate the EM signal to be transmitted by at least one antenna into the body part of the subject. The response resulting from the EM signal interacting with the subject's blood in the body part of the subject may be received by at least one antenna.

In some embodiments, the EM signal may have a wavelength falling within a radiofrequency (RF) waveband or a microwave waveband.

According to a second specific aspect, there is provided a computer-implemented method of training a predictive model, the method comprising acquiring first measurement data indicative of a response resulting from an electromagnetic (EM) signal interacting with a subject's blood in a body part of the subject: acquiring second measurement data indicative of at least one parameter associated with the subject: creating training data from the first measurement data and the second measurement data; and training the predictive model to determine a concentration of glucose in a subject's blood, wherein the predictive model is trained using the training data.

According to third specific aspect, there is provided an apparatus for determining a concentration of glucose in a subject's blood, the apparatus comprising a processor configured to perform steps of any of the methods disclosed herein.

In some embodiments, the apparatus may further comprise a first sensor for sensing the first measurement data.

The first sensor may comprise an antenna configured to receive the response resulting from an electromagnetic (EM) signal interacting with the subject's blood in a body part of the subject.

The apparatus may, in some embodiments, further comprise at least one second sensor for sensing the second measurement data.

The at least one second sensor may comprise a sensor selected from a group comprising: a skin surface temperature sensor: an ambient temperature sensor; a skin moisture level sensor: a humidity sensor: an accelerometer and an inertial measurement unit (IMU).

In some embodiments, the apparatus may further comprise a signal generator configured to generate the electromagnetic (EM) signal. The apparatus may further comprise an antenna in communication with the signal generator, the antenna configured to transmit the EM signal into the body part of the subject.

According to a fourth specific aspect, there is provided a wearable device configured to be worn on a body part of a subject, the wearable device comprising an apparatus as disclosed herein; and an attachment mechanism configured to attach the wearable device to the body part of the subject such that the apparatus is maintained in contact with the subject's skin.

According to a fifth specific aspect, there is provided a system for determining a concentration of glucose in a subject's blood, the system comprising a processor configured to perform steps of any of the methods disclosed herein. The system may further comprise a wearable device configured to be worn on the body part of the subject. The wearable device may comprise a first sensor for sensing the first measurement data: at least one second sensor for sensing the second measurement data; and an attachment mechanism configured to attach the wearable device to the body part of the subject such that the wearable device is maintained in contact with the subject's skin.

These and other aspects will be apparent from and elucidated with reference to the embodiment(s) described hereinafter.

### BRIEF DESCRIPTION OF THE DRAWINGS

Exemplary embodiments will now be described, by way of example only, with reference to the following drawings, in which:

FIG. 1 is a schematic illustration of an example of a device capable of transmitting and receiving an electromagnetic (EM) signal;

FIG. 2 is a flowchart of an example of a method of determining a concentration of glucose in a subject's blood:

FIG. 3 is a flowchart of an example of a further method of determining a concentration of glucose in a subject's blood:

FIGS. 4A-4B show two graphs of example response signals:

FIG. 5 is a flowchart of an example of a method of training a predictive model:

FIG. 6 is a schematic illustration of an example of an apparatus for determining a concentration of glucose in a subject's blood:

FIG. 7 is a schematic illustration of a further example of an apparatus for determining a concentration of glucose in a subject's blood:

FIG. 8 is an illustration of a wearable device:

FIG. 9 is a schematic illustration of a system for determining a concentration of glucose in a subject's blood; and

FIG. 10 is a plot of an example data set comparing a predicted glucose concentration and an actual glucose concentration.

### DETAILED DESCRIPTION OF EMBODIMENTS

Embodiments disclosed herein provide a mechanism by which a person's blood glucose level can be determined using measurements acquired in respect of the person in a non-invasive manner. As used herein, the terms “subject” and “person” are used interchangeably, and include any subject whose blood glucose concentration might be determined, including human and animal subjects.

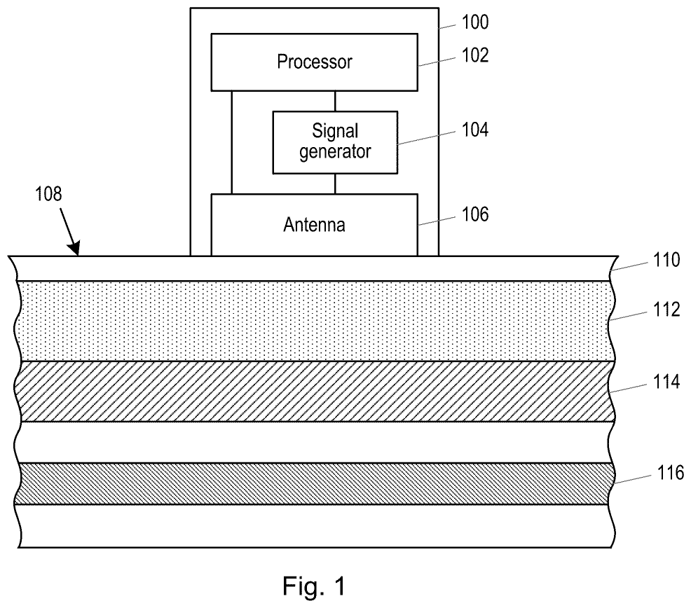

Referring to the drawings, FIG. 1 is a schematic illustration of an example of a device 100 capable of transmitting and receiving an electromagnetic (EM) signal. The device 100 includes a processor 102, and EM signal generator 104 and an antenna 106. Both the EM signal generator 104 and the antenna 106 are in operative communication with the processor 102, and the EM signal generator is in operative communication with the antenna. As shown in FIG. 1, the device 100 may be positioned next to a body part 108 into which it is intended to transmit EM radiation. The body part 108 may be any body part of a subject, such as an arm, a leg, a finger, and earlobe or the like. In the example shown in FIG. 1, the device 100 is positioned such that it is touching a wrist of the subject.

The processor 102 may be configured to operate the EM signal generator 104 to cause the EM signal generator to generate an EM signal which may, for example, comprise a signal having a wavelength in the microwave waveband and/or in the radiofrequency (RF) waveband. The EM signal generated by the EM signal generator 104 is transmitted by the antenna 106 into the body part (e.g., the wrist) 108 of the subject. The body part 108 includes a layer of skin 110 and, depending on the body part, may include tissue 112, bone 114, and blood vessels, veins and/or arteries 116 carrying blood through the body part.

In this example, the antenna 106 is configured to receive a response (e.g., a response signal) resulting from interactions of the transmitted EM signal with components of the body part 108 that the EM signal engages. For the purpose of determining a blood glucose concentration, interactions of the transmitted EM signal with blood in the blood vessels, veins and/or arteries 116 are of particular relevance. In reality, the EM signal reflects off everything it encounters in the body part, including skin 110, tissue 112, bone 114 and blood. Reflections from the skin 110, the tissue 112 and the bone 114 remain generally constant over time, although they may vary under different conditions (e.g., if the skin is sweaty). The response signal resulting from interactions of the EM signal with blood changes with variations in the blood glucose level and, therefore, changes in the response signal resulting from changes in blood glucose levels can be used to determine a concentration of blood glucose in the blood.

In the example shown in FIG. 1, and in other embodiments disclosed herein, the response signal received and analysed results from the reflection of the transmitted EM signal from components in the body part 108. Thus, the antenna 106 may function as both a transmit antenna and a receive antenna. Alternatively, a transmit antenna may be provided to transmit the EM signal into the body part, and a receive antenna may be provided next to, or within, the transmit antenna, to receive the reflected EM signal (e.g., the response signal). In other examples, a transmit antenna may be positioned one side of the body part and a receive antenna may be positioned at an opposite side of the body part, configured to receive a response signal based on the EM signal that is able to pass through the body part. In both cases (i.e., when the response signal is based on a reflected signal or a signal transmitted through the body part), the response signal is based on interactions between the EM signal and blood in the body part.

The device 100 and/or the components thereof may form the basis of components used to acquire measurements used in further examples described herein.

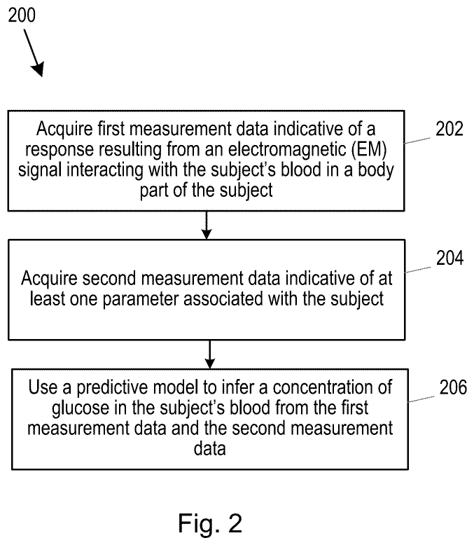

FIG. 2 is a flowchart of an example of a method 200, such as a computer-implemented method, of determining a concentration of glucose in a subject's blood. The method 200 comprises, at step 202, acquiring first measurement data indicative of a response resulting from an electromagnetic (EM) signal interacting with the subject's blood in a body part of the subject. The EM signal may be generated using any device suitable for generating EM signals to be transmitted into a subject's body part for the purpose of determining a concentration of glucose in the blood. For example, the EM signal may be generated using the device 100 described above with reference to FIG. 1.

As noted above, the EM signal that interacts with the subject's blood in the body part may comprise an EM signal having a wavelength or frequency falling within the radiofrequency (RF) waveband and/or a wavelength falling within the microwave waveband. In some embodiments, the EM signal may have a frequency in the range 200 kHz to 10 GHz. In some embodiments, the EM signal may have frequency in the range 4.5 GHz to 7.5 GHz or, more preferably, in the range 5 GHz to 7 GHz. This range is particularly relevant for determining changes resulting from glucose in the blood. In reality, the EM signal may be scanned over a range of frequencies within the wavebands/ranges mentioned above.

It may be possible to determine an indication of a concentration of glucose in the subject's blood from the first measurement data acquired at step 202. For example, one such technique is disclosed in WO2017/141024, filed in the name of Orsus Medical Ltd, and on which the present invention builds. As noted above, several factors affect the response signal in addition to the concentration of glucose. For example, parameters associated with the subject may affect the response, including for example environmental parameters or conditions, and/or parameters relating to how the device (e.g., the device 100) is positioned relative to the subject. Thus, the present disclosure acquires additional data that is used to take account of changes in the response signal resulting from environmental conditions and/or parameters other than the concentration of glucose in the subject's blood. It is noted that the term “environmental” used herein is intended to refer to the environment associated with the subject.

At step 204, the method 200 comprises acquiring second measurement data indicative of at least one parameter associated with the subject. The second measurement data may, for example, be acquired using one or more sensors. In some examples, each sensor may be configured to measure data indicative of a respective parameter associated with the subject while, in other examples, data indicative of multiple parameters may be measured using one sensor. The at least one parameter associated with the subject may comprise a parameter selected from a group comprising: a temperature of a surface of skin of the body part: an ambient temperature around the body part: a level of moisture on a surface of skin of the body part: an ambient humidity around the body part; and a level of motion of the body part.

The temperature of a surface of skin of the body part may, for example, be measured using a first temperature sensor, such as a thermistor or an infrared temperature sensor. The sensor used to measure the temperature of the surface of the skin may, for example, be positioned in contact with the skin at or near to where the EM signal is transmitted into the body part.

The ambient temperature around the body part may, for example, be measured using a second temperature sensor, such as a thermistor or a thermocouple. The sensor used to measure the ambient temperature around the body part may, for example, be positioned near to, but not touching, the body part.

The level of moisture on a surface of the skin of the body part may, for example, be measured using a capacitive sensor or some other moisture meter. The sensor used to measure the level of moisture on this surface of the skin may, for example, be positioned in contact with the skin at or near to where the EM signal is transmitted into the body part. The level of moisture on the skin surface can vary if the subject allows the body part to get wet before the EM signal is transmitted into the body part and/or if sweat has accumulated on the body part.

The ambient humidity around the body part may, for example, be measured using a humidity sensor, such as a capacitive humidity sensor or a hygrometer. The sensor used to measure the ambient humidity around the body part may, for example, be positioned near to, but not touching, the body part.

The level of motion of the body part may, for example, be measured using an accelerometer or an inertial measurement unit (IMU) which may include one or more accelerometers, gyroscopes and/or magnetometers. The level of motion of the body part may include an indication of a direction and/or speed of motion of the body part around the time that the EM signal is transmitted into the body part. The sensor used to measure the level of motion of the body part may, for example, remain in a constant position relative to the body part, such that the measured level of motion of the sensor is indicative of the level of motion of the body part.

The method 200 comprises, at step 206, using a predictive model to infer a concentration of glucose in the subject's blood from the first measurement data and the second measurement data, the predictive model having been trained to infer a concentration of glucose in the subject's blood from the first measurement data and the second measurement data. In other words, the predictive model-sometimes referred to as a prediction model-receives, as its inputs, the first measurement data indicative of a response resulting from an EM signal interacting with the subject's blood and the second measurement data indicative of at least one parameter associated with the subject. The predictive model is trained to infer or determine, based on those inputs, a concentration of glucose in the subject's blood.

In some embodiments, the predictive model may comprise a machine learning model or algorithm. Any appropriate machine learning models or techniques may be implemented, including for example artificial neural networks, deep neural networks, random forest models, regression trees, classifiers, and the like. In one example, a trained artificial neural network may be used to infer the concentration of glucose in the subject's blood from the inputs.

Artificial neural networks or, simply, neural networks, will be familiar to those skilled in the art, but in brief, a neural network is a type of model that can be used to annotate (for example, classify or label) data or infer data based on its inputs (for example, determine a concentration of glucose based on an EM response signal and a parameter associated with the subject). The structure of a neural network is inspired by the human brain. Neural networks are comprised of layers, each layer comprising a plurality of neurons. Each neuron comprises a mathematical operation. In the process of classifying data, the mathematical operation of each neuron is performed on the data to produce a numerical output, and the outputs of each layer in the neural network are fed into the next layer sequentially. The magnitude of the numerical output of a neuron (when classifying data) is often referred to as the “activation level” of that neuron. In some neural networks, such as convolutional neural networks, lower layers in the neural network (i.e., layers towards the beginning of the series of layers in the neural network) are activated by (i.e., their output depends on) small features or patterns in the data being classified, while higher layers (i.e., layers towards the end of the series of layers in the neural network) are activated by increasingly larger features in the data being classified. As an example, where the input data comprises data from an EM response signal and parameter data associated with the subject, and the model comprises a neural network, different parts of the input data may active different layers in the neural network. Data of different classifications create different activation patterns (e.g., have different activation signatures in the network). A neural network thus classifies data according to the activation pattern produced in the neural network.

In some examples herein, where the input data comprises the first measurement data and the second measurement data discussed above, and the model is for determining a blood glucose concentration, each neuron in the neural network may comprise a mathematical operation comprising a weighted linear sum of part of the input data, followed by a non-linear transformation. Examples of non-linear transformations used in neural networks include sigmoid functions, the hyperbolic tangent function and the rectified linear function. The neurons in each layer of the neural network generally comprise a different weighted combination of a single type of transformation (e.g., the same type of transformation, sigmoid etc. but with different weightings). As will be familiar to the skilled person, in some layers, the same weights may be applied by each neuron in the linear sum: this applies, for example, in the case of a convolutional layer. The output of each neuron may be a number and as noted above, the magnitudes of the numerical outputs of the neurons form a neuron activation pattern that may be used to classify the image.

Generally, such a neural network model may comprise any type of neural network model that can be used to annotate (e.g., classify) data. Examples of models include, but are not limited to feed forward models (such as convolutional neural networks, autoencoder neural network models, probabilistic neural network models and time delay neural network models), radial basis function network models, recurrent neural network models (such as fully recurrent models, Hopfield models, or Boltzmann machine models), or any other type of neural network model.

In an example trial using a neural network model, an 80/20 model building approach was used, where the model was fitted, and weights were adjusted using the left out 20% of training data until the model converged. The model was then used on a trial data set left out of the analysis. The output of the neural network model—and other predictive models—is assessed using a calculation of a Mean Absolute Relative Deviation (MARD) of the predicted glucose concentration, compared to the observed or measured glucose concentration determined using a “gold standard” approach (e.g., a measurement taken from blood of the subject). The lower the MARD value, the more accurate the prediction (i.e., the inferred glucose concentration from the predictive model).

Table 1 below shows MARD values calculated for a set of subjects using a trained neural network.

TABLE 1

Session = Subject/date ID

| Train | Test | Subject | MARD % |
|---|---|---|---|
| All - 1 | 1 | 1 | 9.64 |
| All - 2 | 2 | 1 | 11.92 |
| All - 3 | 3 | 1 | 9.96 |
| All - 5 | 5 | 2 | 40.43 |
| All - 6 | 6 | 2 | 23.64 |
| All - 7 | 7 | 2 | 11.09 |
| All - 8 | 8 | 3 | 20.99 |
| All - 9 | 9 | 3 | 11.45 |
| All - 10 | 10 | 4 | 8.83 |
| All - 14 | 14 | 5 | 13.16 |
| All - 15 | 15 | 5 | 14.09 |
| All - 16 | 16 | 3 | 21.29 |
| All - 17 | 17 | 1 | 11.03 |
| All - 18 | 18 | 5 | 17.16 |
| All - 20 | 20 | 3 | 7.17 |
| All - 21 | 21 | 6 | 10.31 |
| All - 22 | 22 | 7 | 10.33 |
| All - 23 | 23 | 8 | 8.87 |
| All - 24 | 24 | 6 | 10.47 |
| All - 25 | 25 | 7 | 9.38 |
| All - 26 | 26 | 9 | 10.20 |
| All - 27 | 27 | 8 | 12.24 |
| All - 28 | 28 | 10 | 9.21 |
| All - 29 | 29 | 6 | 11.52 |
| All - 30 | 30 | 9 | 30.80 |
| All - 31 | 31 | 10 | 6.10 |
| All - 32 | 32 | 7 | 12.87 |
| All - 33 | 33 | 8 | 19.26 |
| All - 34 | 34 | 9 | 9.09 |
| All - 35 | 35 | 10 | 8.76 |
| All - 36 | 36 | 7 | 7.94 |
| All - 37 | 37 | 8 | 12.93 |
| All - 38 | 38 | 9 | 10.04 |
| All - 39 | 39 | 10 | 9.74 |

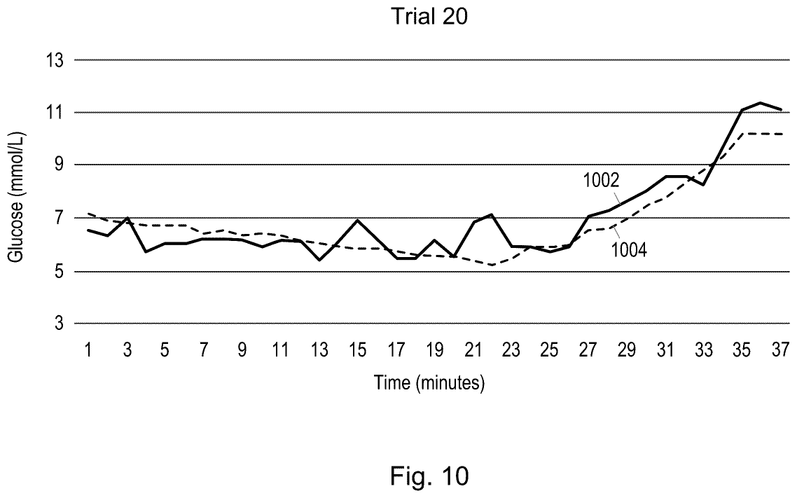

Turning to FIG. 10, a plot is shown for an example data set for one subject. In the plot, a line 1002 represents the predicted (e.g., inferred) glucose concentration provided by the predictive model, and a line 1004 represents the actual glucose concentration determined using a “gold standard” method. In the example shown, the MARD is calculated as 7.1%.

In another example, a trained random forest model may be used to infer the concentration of glucose in the subject's blood from the inputs. A random forest model is a type of machine learning algorithm that can be used for both classification and regression tasks. The random forest model may seek to establish a relationship between the input features (e.g., the first and second measurement data) and a target variable which, in this case, is the concentration of glucose in the subject's blood. In some embodiments, data for multiple subject s (e.g., training subjects) may be provided as training data for the random forest model. For each subject, first measurement data (e.g., EM response signal, or the frequency of the minimum in the response signal, or a combination of the frequency and the amplitude of the minimum in the response signal) and second measurement data (e.g., the skin temperature, the ambient temperature, the skin moisture level, the ambient humidity and an indication of the motion of the body part) may be provided. In some examples, data indicative of actual glucose concentration measurements for a subject (e.g., obtained through a finger prick test or using an implanted glucose measurement device) may also be provided in the training data, and this can be used in supervised training of the model.

In an example trial using a random forest model, a nested cross validation approach was used to make for convergence.

Used in a regression task, the random forest model may aim to approximate the underlying function that maps the input variables to the output variable.

The random forest model may include multiple decision trees, each of which may be trained on a random subset of the training data.

Determining a concentration of glucose in the subject's blood using the predictive model (e.g., a machine learning model such as a random forest model) can be more accurate than determining the glucose concentration from the EM response signal using existing techniques that do not use machine learning, and/or do not make use of the second measurement data used by the predictive model.

In another example, a partial least squares model was used, and using this model, an approach involving 10 times cross validation of the model was used to look for convergence, before testing the fit.

In another example, a multiple linear regression model was used, and for this model, a forward backward approach for model building was used, with an adjusted R2 criteria.

The skilled person will be aware of other types of models that the teachings herein will apply to.

In the examples above, training of the predictive models was achieved using a ‘leave one trial out’ (LOTO) or a ‘leave one subject out’ (LOSO) approach. The LOTO approach takes the data set, puts a single trial aside, and uses the remaining data to train the predictive model/algorithm. For the neural network, the remaining data set was split into 80%/20% where the 80% is the fit data and the 20% is the test data. The fit data estimates the model parameters, and the model is then tested on the 20% test data set. Based on that fit, the model's weights are updated via a gradient method, and then the process is repeated until a model is converged after multiple iterations. That model then predicts the single trial left out to calculate a MARD for that trial. This process is repeated for every trial as an estimate of the overall MARD of the system. Similarly, the LOSO approach determines a MARD for a new subject who wears the device for the first time with no calibration.

An example of the training data used to train the predictive models is shown in Table 2 below.

TABLE 2

| | |
|---|---|
| Trial | 1 |
| Subject | 1 |
| Date | Aug. 7, 2022 |
| Device | 2420 3_25 |
| Age | 56 |
| Sex | M |
| Diabetes Type | T2 |
| Sample Num | 15 |
| DateTime | 08:50 |
| Delta Time | 00:00 |
| Order Num | 1 |
| R3-GM | 5117637656 |
| R3-GM ADC | −24.26989908 |
| R3-GM-Clean | 5118488855 |
| R3-GM ADC-Clean | −24.66899072 |
| R3-Humidity FreeSpace | 88.44604492 |
| R3-Humidity Skin | 64.30358887 |
| R3-Start Temp | 30.0390625 |
| R4-GM | 7068295583 |
| R4-GM ADC | −37.45969006 |
| R4-GM-Clean | 7068521940 |
| R4-GM ADC-Clean | −37.96844275 |
| R4-Humidity FreeSpace | 89.19067383 |
| R4-Humidity Skin | 64.42260742 |
| R4-Start Temp | 30.0546875 |
| Glucose | 10.13 |

In Table 2, the fields starting R3 and R4 relate to the first and second measurement data.

The first measurement data and/or the second measurement data may be processed, manipulated and/or formatted prior to being provided as inputs to the predictive model. For example, the first/second measurement data may be formatted in such a way that the inputs of the predictive model are dimensionless.

Similarly, the output of the predictive model, which may comprise a dimensionless value, may be processed, manipulated and/or formatted such that it is representative or indicative of the concentration of glucose in the blood of the subject in respect of whom the first and second measurement data was acquired.

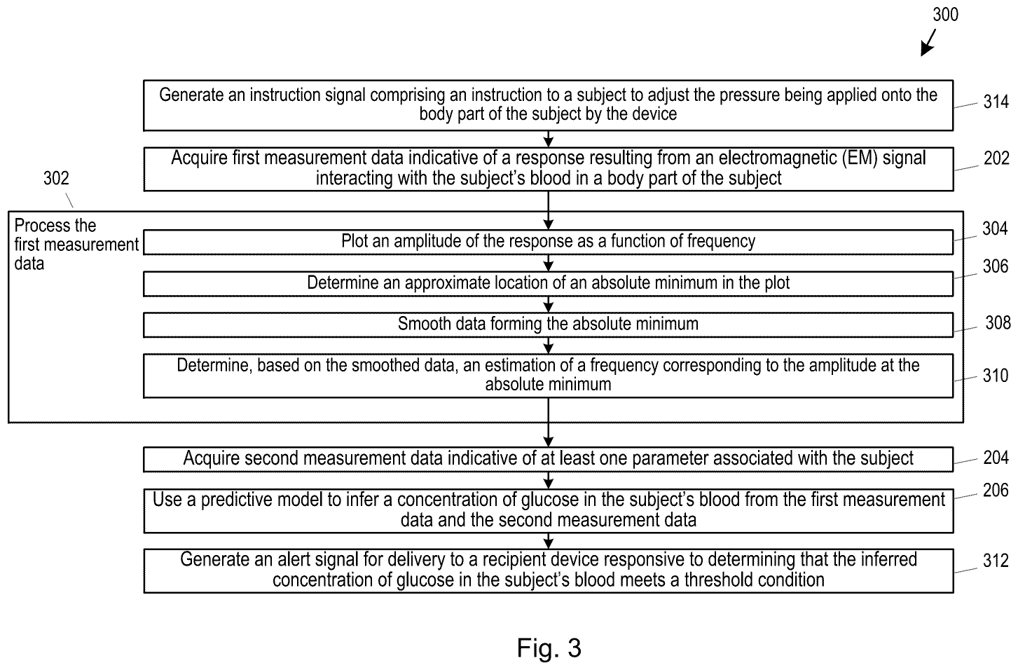

FIG. 3 is a flowchart of a further example of a method 300 of determining a concentration of glucose in a subject's blood. The method 300 may include steps of the method 200 discussed above. The method 300 may further comprise, at step 302, processing the first measurement data. In other words, at step 302, the response resulting from the EM signal interacting with the subject's blood may be processed. In some examples, the processed first measurement data may be provided to the predictive model as an input.

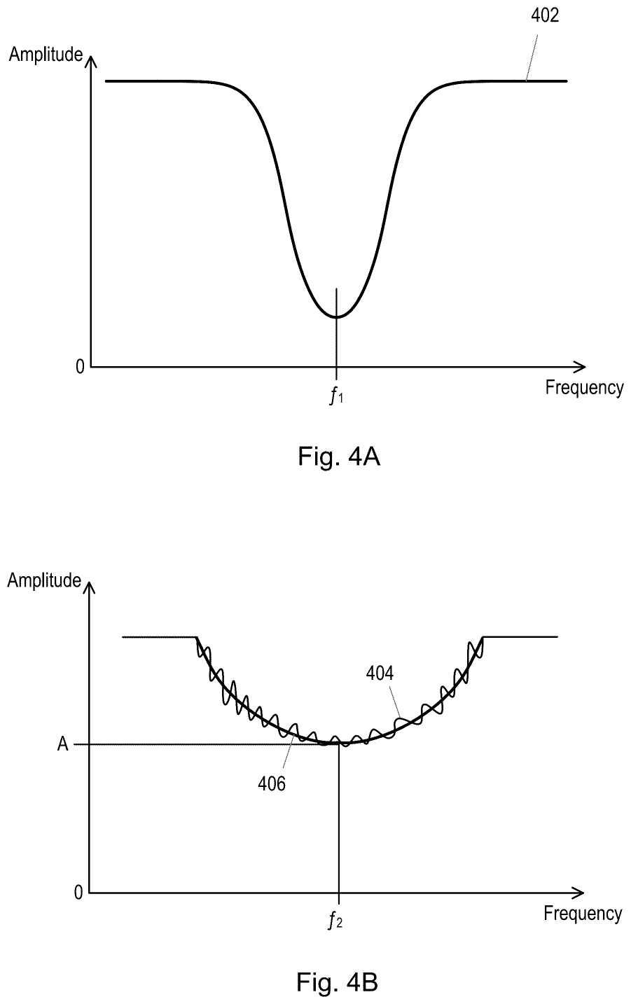

The processing of the first measurement data (step 302) is discussed with reference to FIGS. 4A and 4B, which are graphs showing an example responses obtained at step 202. The step of processing the first measurement data may include, at step 304, plotting an amplitude of the response as a function of frequency, for example as shown in FIG. 4A. At step 306, the processing 302 may include determining an approximate location of an absolute minimum in the plot. In a response with no noise, the plot may resemble that shown by line 402 in FIG. 4A, with a smooth plot and a clearly defined absolute minimum at a frequency f₁ along with a corresponding amplitude (e.g., having the units dB). However, in reality, noise resulting from temperature fluctuations, changes in the level of moisture on the subject's skin, movement of the body part as the response is acquired, and the like, can lead to a less defined response signal, such as that shown by line 404 in FIG. 4B. This case, it can be more difficult to determine the frequency of the absolute minimum accurately, and the processing steps 304 to 310 can help with that determination. In some cases, noise caused by interference and/or crosstalk from other electronic devices (e.g., mobile telephones and WiFi routers) may also contribute to noise in the response signal. Thus, in some embodiments, a noise cancellation operation may be performed to reduce the noise in the response signal.

At step 308, the processing 302 may include smoothing data forming the absolute minimum. By performing a smoothing operation on the data that forms the minimum, or on the data around the minimum, it is possible to remove the small peaks and troughs caused by noise, making it possible to determine more accurately the true frequency and corresponding amplitude at the minimum. Without the smoothing operation, the minimum may be less clear, and it may therefore be more difficult to accurately determine the frequency at the minimum. As a result of the smoothing operation, the plot or curve may be smoother, for example resembling the line 406 shown in FIG. 4B. In this way, it is possible to determine more accurately an estimation of the frequency f₂ at which the absolute minimum occurs, and the corresponding amplitude A. Thus, at step 310, the processing 302 may include determining, based on the smoothed data, an estimation of a frequency corresponding to the amplitude at the absolute minimum. In some embodiments, the processing may further include determining, based on the smoothed data, an estimation of an amplitude corresponding to the frequency at the absolute minimum.

It should be noted that, in some cases, the step of processing 302 may not be required, for example if the response signal does not suffer from high levels of noise. Whether or not the step of processing 302 is performed, the frequency and optionally the corresponding amplitude of an absolute minimum in the response signal may, in some cases, be provided as an input (e.g., as the first measurement data) to the predictive model. Thus, in some embodiments, the first measurement data may comprise data indicative of a frequency corresponding to an absolute minimum in the response resulting from the EM signal interacting with the subject's blood in the body part of the subject. In some examples, the value of the amplitude at the absolute minimum may also be provided as part of the first measurement data to the predictive model. It is also noted that the processing of step 302 may be performed before or after step 204.

The concentration of glucose in the subject's blood provided as an output by the predictive model may be provided for delivery or presentation to a recipient, such as the subject. In some embodiments, the glucose concentration may be stored in a memory, such as a memory in a device accessible by the subject and/or in a cloud-based memory. The glucose concentration may be provided to a recipient other than the subject, such as a medical professional (e.g., a doctor). In this way, in the event that the inferred glucose concentration is particularly high or particularly low (e.g., outside a defined range) then the medical professional can be made aware in a timely manner, and may be able to take appropriate action.

In some examples, delivery of the glucose concentration or of some other message or signal to a recipient may be conditional on the value of the inferred or determined glucose concentration. Thus, in some embodiments, the method 300 may comprise, at step 312, generating an alert signal for delivery to a recipient device responsive to determining that the inferred concentration of glucose in the subject's blood meets a threshold condition. For example, if it is determined that the inferred concentration of glucose in the subject's blood meets or exceeds a defined maximum safe threshold, then an alert signal may be generated and delivered to a recipient device. Similarly, if it is determined that the inferred concentration of glucose in the subject's blood meets or is lower than a defined minimum safe threshold, then an alert signal may be generated and delivered to a recipient device. The recipient device may comprise a device in the possession of the subject and/or a device in the possession of a medical professional. In this way, if the subject is made aware that their blood glucose level is outside of a defined safe range, then they can take immediate action to bring their blood glucose level into the safe range.

As noted above, the EM signal transmitted into the subject's body part, from which the response signal is acquired, may have a frequency in a range from around 200 kHz to 10 GHz. In some embodiments, multiple EM signals may be transmitted into the subject's body part, and multiple response signals may be acquired. For example, a first EM signal may be generated having a first frequency, and interactions of the first EM signal with blood in the subject's body part may give rise to a first response signal. A second EM signal may be generated having a second frequency, and interactions of the second EM signal with blood in the subject's body part may give rise to a second response signal. Thus, the first measurement data may comprise: a) data indicative of a response resulting from an EM signal in a first EM waveband interacting with the subject's blood in the body part of the subject; and b) data indicative of a response resulting from an EM signal in a second EM waveband interacting with the subject's blood in the body part of the subject. In examples where multiple response signals are acquired (e.g., from EM signals into different EM wavebands) then data indicative of both the response signals may be provided as inputs into the predictive model.

Various EM wavebands may be relevant for determining a blood glucose concentration in a subject's blood, for example wavebands around 5 GHz and 6.5 GHz. Generally, the first EM waveband and the second EM waveband both fall within a range of between 200 kHz and 10 GHz. In some examples, the first and second EM wavebands may fall within a range of approximately 4.5 GHz to 7 GHz, in other examples, the EM signal in the first waveband may have a frequency of between 4.5 GHz and 6 GHz, and the EM signal in the second waveband may have a frequency of between 6 GHz and 7.5 GHz and, in yet further examples, the EM signal in the first waveband may have a frequency of between 4.8 GHz and 5.4 GHz, and the EM signal in the second waveband may have a frequency of between 6.3 GHz and 6.7 GHz. As noted above, transmitting the EM signal into the body part may comprise scanning the EM signal over a range of frequencies, for example a range of frequencies in the first waveband and/or the second waveband.

As discussed above, the EM signal interacts with various items within the subject's body part (including the blood), and these interactions can be detected in a response signal acquired either from transmission through the body part or from reflection off items in the body part. Thus, in some embodiments, the first measurement data may comprise data indicative of a response resulting from the EM signal reflecting from the subject's blood as the EM signal interacts with the body part of the subject.

In addition to acquiring (e.g., receiving) the first and second measurement data to be used by the predictive model, the method 300 may also include steps relating to generating the EM signal to be transmitted into the body part. For example, at step 314, the method 300 may further comprise, prior to acquiring the first measurement data, controlling a signal generator to generate the EM signal to be transmitted by at least one antenna into the body part of the subject. The response resulting from the EM signal interacting with the subject's blood in the body part of the subject may be received by at least one antenna. In some examples, the same antenna that is used to transmit the EM signal into the body part may also be used to receive the response resulting from EM signal interacting with the subject's blood. In other examples, multiple antennas may be provided, such as a first antenna to transmit the EM signal and a second antenna to receive the response signal.

The method 200, 300 may be repeated (e.g., periodically) at regular or irregular intervals over a period of time so that the subject's blood glucose level can be monitored and any particularly high or low levels can be quickly identified and acted upon. For example, first and second measurement data may be acquired every 30 seconds, 1 minute, 2 minutes, 5 minutes, 10 minutes, or the like, and each time measurement data is acquired, it may be provided to the predictive model to infer a blood glucose concentration. In this way, the method 200, 300 may be considered to be methods of continuous glucose monitoring.

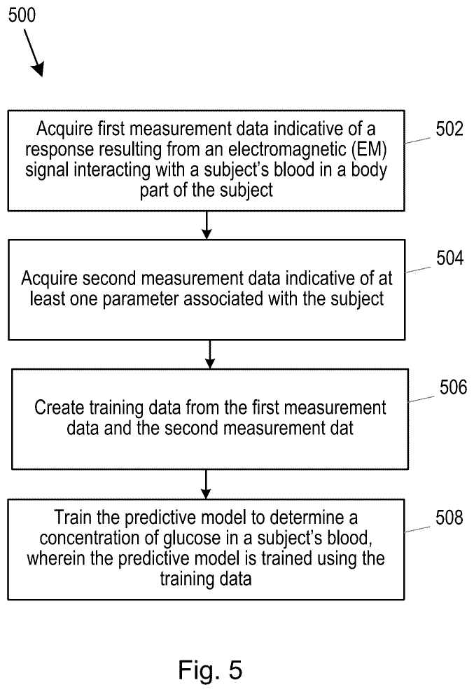

FIG. 5 is a flowchart of an example of a method 500 of training a predictive model. For example, the predictive model trained using the method 500 may comprise of the predictive model used in the methods 200, 300 discussed above. The method 500, which may comprise a computer-implemented method, comprises, at step 502, acquiring first measurement data indicative of a response resulting from an electromagnetic (EM) signal interacting with a subject's blood in a body part of the subject. In some examples, the first measurement data may be acquired (step 502) using methods similar to the methods used to acquire the first measurement data in step 202 discussed above. At step 504, the method 500 comprises acquiring second measurement data indicative of at least one parameter associated with the subject. Similarly, the second measurement data may, in some examples, be acquired (step 504) using methods similar to the methods used to acquire the second measurement data in step 204 discussed above.

The method 500 comprises, at step 506, creating training data from the first measurement data and the second measurement data. The training data created at step 506 may include the acquired first and second measurement data formatted in a format suitable for training the predictive model. The training data may, for example, include first and second measurement data acquired in respect of multiple different subjects and/or first and second measurement data acquired at multiple different times from the same subject. In some examples, the second measurement data used to create the training data may include measurement data indicative of just one parameter associated with the subject (e.g., skin temperature) while, in other examples, the second measurement data used to create the training data may include measurement data indicative of all of the parameters associated with the subject discussed herein. The training data may, in some embodiments, further include additional information, such as an actual measurement of the concentration of glucose in a subject's blood. Such an actual measurement may be acquired from a finger prick test, for example. An advantage of providing actual measurements as part of the training data is that the predictive model can learn which first measurement data and second measurement data give rise to a particular blood glucose concentration. In this way, the blood glucose level may be monitored regularly in a non-invasive manner, and the subject may not even be aware that measurements are being acquired.

The method 500 comprises, at step 508, training the predictive model to determine a concentration of glucose in a subject's blood, wherein the predictive model is trained using the training data. The predictive model may be trained using known training techniques, such as those used to train a machine learning models, such as neural networks.

As noted above, any of the methods 200, 300, 500 disclosed herein may be computer-implemented methods, such that any of the steps of those methods may be performed using one or more processors or processing circuitry. In some examples, all of the steps of a method may be performed using a single processor while, in other examples, different processes may be used to perform different steps of the method. One or more processors used to perform steps of the methods may form part of a computing device (e.g., a wearable device, a smart phone, a tablet computer or a desktop computer) or part of a server in a cloud-computing environment.

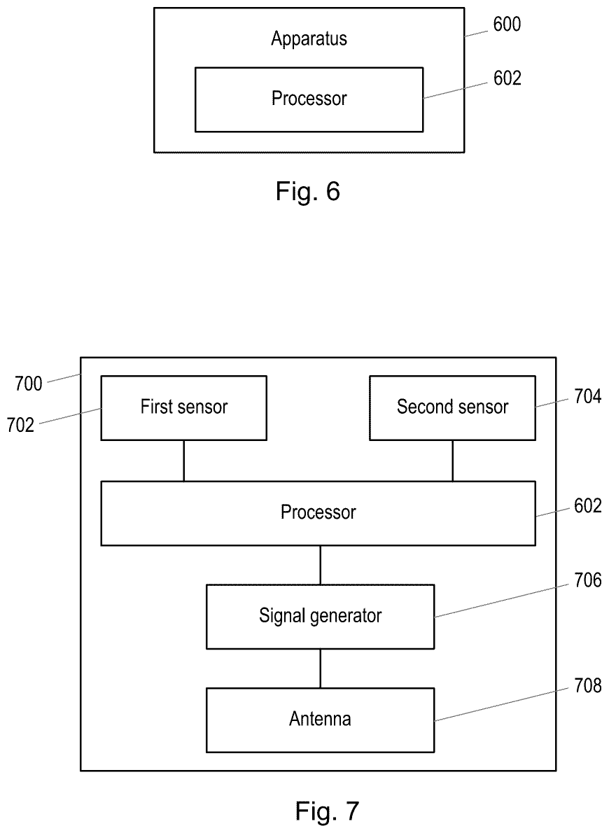

FIG. 6 is a schematic illustration of an example of an apparatus 600 for determining a concentration of glucose in a subject's blood. The apparatus 600 comprises a processor 602 configured to perform steps of any of the methods 200, 300, 500 disclosed herein. In some examples, the apparatus 600 may further include a machine-readable medium comprising instructions which, when executed by a processor, such as the processor 602, cause the processor to perform steps of any of the methods 200, 300, 500 disclosed herein. In other examples, the machine-readable medium may not form part of the apparatus 600, but may be in operative communication with the processor 602.

FIG. 7 is a schematic illustration of a further example of an apparatus 700 for determining a concentration of glucose in a subject's blood. The apparatus 700 may comprise a processor, such as the processor 602. The apparatus 700 may, in some embodiments, further comprise a first sensor 702 for sensing the first measurement data. In some embodiments, the apparatus 700 may further comprise at least one second sensor 704 for sensing the second measurement data. The first sensor 702 and/or the at least one second sensor 704 may be in operative communication with the processor 602.

The first sensor 702 may comprise an antenna configured to receive the response resulting from an electromagnetic (EM) signal interacting with the subject's blood in a body part of the subject. In some examples, the antenna may also be used to transmit the EM signal into the body part of the subject. The at least one second sensor 704 may comprise a sensor selected from a group comprising: a skin surface temperature sensor: an ambient temperature sensor: a skin moisture level sensor: a humidity sensor: an accelerometer and an inertial measurement unit (IMU). For example, multiple second sensors 704 may be included in the apparatus 700, each second sensor 704 configured to measure data indicative of a different parameter (e.g., skin surface temperature, ambient temperature, a skin moisture level, ambient humidity, and motion of the body part).

The apparatus 700 may, in some embodiments, further comprise a signal generator 706 configured to generate the electromagnetic (EM) signal. The apparatus 700 may further comprise an antenna 708 in communication with the signal generator 706, be antenna configured to transmit the EM signal into the body part of the subject. The antenna 708 may comprise antenna configured in any suitable manner for transmitting and EM signal (e.g., an RF signal or microwave signal) into a body part of a subject for the purposes disclosed herein.

In some embodiments, the apparatus 700 may further comprise a user interface (e.g., a display) (not shown) configured to display the inferred concentration of glucose to a user. In this way, a user (e.g., the subject) may receive a real time update of their blood glucose level.

The apparatus 700 may, in some examples, include a communications unit (not shown) configured to transmit and/or receive data acquired by and/or to be used by various components of the apparatus. For example, the communications unit may transmit data acquired using one or more of the first sensor 702 and the second sensor 704 to a remote device or processor.

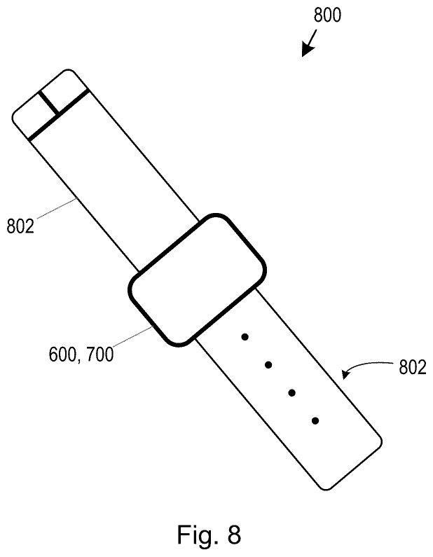

In some embodiments, the apparatus 700 may comprise or form part of a wearable device that can be worn by the subject for the majority of the time, such that regular measurements of the first and second measurement data can be acquired and used to determine a concentration of glucose in the subject's skin. FIG. 8 is an illustration of an example of a wearable device 800. The wearable device 800 is configured to be worn on a body part of a subject. In the example shown in FIG. 8, the wearable device 800 may be worn on a wrist of the subject. The wearable device 800 comprises an apparatus 600, 700 as disclosed herein. The wearable device 800 further comprises an attachment mechanism 802 configured to attach the wearable device to the body part of the subject such that the apparatus 600, 700 is maintained in contact with the subject's skin. In this example, the attachment mechanism 802 comprises a strap having a buckle that can be tightened and secured around the subject's wrist, such that the apparatus 600, 700 is maintained in contact with the skin of the wrist. In other examples, a different attachment mechanism 802 may be provided.

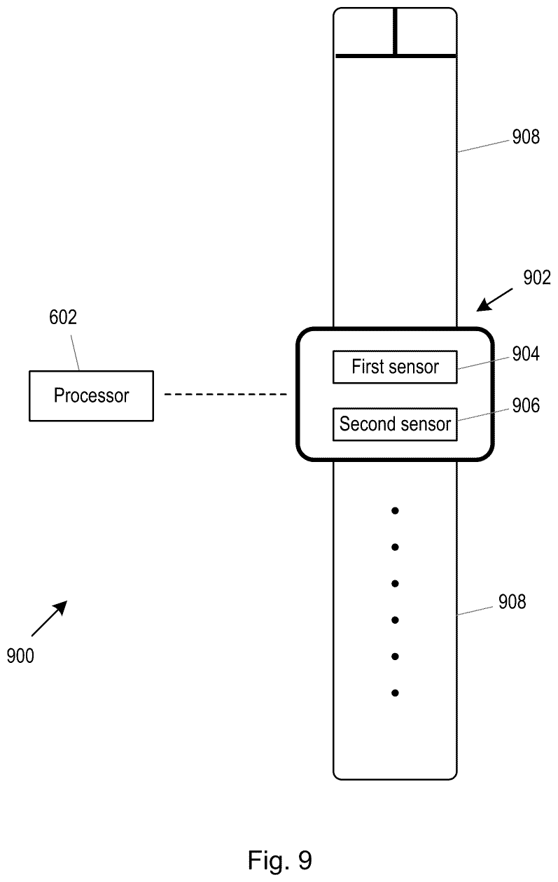

In the apparatus 600, 700 discussed above, the processor 602 is contained within the same unit (i.e., the apparatus) as the other components. According to a further aspect, the processor 602 may be located remotely with respect to the first sensor 702 and the at least one second sensor 704 and/or with other components of the apparatus 600, 700. FIG. 9 is a schematic illustration of an example of a system 900 for determining a concentration of glucose in a subject's blood. The system 900 comprises a processor (e.g., the processor 602) configured to perform steps of any one of the method 200 and/or the method 300. The system 900 further comprises a wearable device 902 configured to be worn on the body part of the subject (i.e., the subject in respect of whom the method 200, 300 is to be performed). The wearable device comprises a first sensor 904 for sensing the first measurement data and at least one second sensor 906 for sensing the second measurement data. The wearable device 902 further comprises an attachment mechanism 908 configured to attach the wearable device 902 to the body part of the subject such that the wearable device is maintained in contact with the subject's skin. By separating the wearable device 902 from the processor 602, data acquired by the first sensor 904 and/or the at least one second sensor 906 may be transmitted to the processor for processing (e.g., for providing has inputs to the predictive model). In this way, the processor 602 may be larger and/or more powerful as it may be located in a computing device or server, rather than in the wearable device 902, where space and power constraints are more limited.

The system may further comprise a user interface (not shown) configured to present information to a user. The user interface may, for example, comprise a user interface or display of a computing device such as a smartphone, such that a user (e.g., the subject) can be informed of a glucose concentration exceeding a defined range (e.g., a safe range).

The processor 602 can comprise one or more processors, processing units, multi-core processors or modules that are configured or programmed to control the apparatus 600, 700 and/or components of the apparatus or the wearable device 800, 902 in the manner described herein. In particular implementations, the processor 602 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g., Java interpreter instructions). Alternatively, one or more or all of the sub-routines may be stored in at least one external library file and linked with a main program either statically or dynamically, e.g., at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer-implemented method of determining a concentration of glucose in a subject's blood, the method comprising:
acquiring first measurement data indicative of a response resulting from an electromagnetic (EM) signal interacting with the subject's blood in a body part of the subject:
acquiring second measurement data indicative of at least one parameter associated with the subject:
using a predictive model to infer a concentration of glucose in the subject's blood from the first measurement data and the second measurement data, the predictive model having been trained to infer a concentration of glucose in the subject's blood from the first measurement data and the second measurement data; and
responsive to determining that the inferred concentration of glucose in the subject's blood meets a threshold condition, generating an alert signal for delivery to a recipient device.

2. The computer-implemented method of claim 1, wherein the threshold condition comprises a maximum safe threshold or a minimum safe threshold.

3. The computer-implemented method of claim 1, wherein the first measurement data comprises data indicative of a frequency corresponding to an absolute minimum in the response resulting from the EM signal interacting with the subject's blood in the body part of the subject.

4. The computer-implemented method of claim 1, wherein the EM signal has a frequency in the range 200 kHz to 10 GHz.

5. The computer-implemented method of claim 1, wherein the first measurement data comprises: a) data indicative of a response resulting from an EM signal in a first EM waveband interacting with the subject's blood in the body part of the subject; and b) data indicative of a response resulting from an EM signal in a second EM waveband interacting with the subject's blood in the body part of the subject.

6. The computer-implemented method of claim 5, wherein the EM signal in the first waveband has a frequency of between 4.8 GHz and 5.4 GHz, and the EM signal in the second waveband has a frequency of between 6.3 GHz and 6.7 GHz.

7. The computer-implemented method of claim 1, wherein the first measurement data comprises data indicative of a response resulting from the EM signal reflecting from the subject's blood as the EM signal interacts with the body part of the subject.

8. The computer-implemented method of claim 1, wherein the at least one parameter associated with the subject comprises a parameter selected from a group comprising: a temperature of a surface of skin of the body part; an ambient temperature around the body part: a level of moisture on a surface of skin of the body part: an ambient humidity around the body part; and a level of motion of the body part.

9. The computer-implemented method of claim 1, further comprising:
processing the first measurement data by:
plotting an amplitude of the response as a function of frequency:
determining an approximate location of an absolute minimum in the plot:
smoothing data in the curve of the absolute minimum; and
determining, based on the smoothed data, an estimation of a frequency corresponding to the amplitude at the absolute minimum.

10. The computer-implemented method of claim 1, further comprising, prior to acquiring the first measurement data:
controlling a signal generator to generate the EM signal to be transmitted by at least one antenna into the body part of the subject:
wherein the response resulting from the EM signal interacting with the subject's blood in the body part of the subject is received by at least one antenna.

11. The computer-implemented method of claim 1, wherein the EM signal has a wavelength falling within a radiofrequency (RF) waveband or a microwave waveband.

12. A computer-implemented method of training a predictive model, the method comprising:
acquiring first measurement data indicative of a response resulting from an electromagnetic (EM) signal interacting with a subject's blood in a body part of the subject:
acquiring second measurement data indicative of at least one parameter associated with the subject:
creating training data from the first measurement data and the second measurement data; and
training the predictive model to determine a concentration of glucose in a subject's blood, wherein the predictive model is trained using the training data.

13. An apparatus for determining a concentration of glucose in a subject's blood, the apparatus comprising:
a processor configured to perform steps of a method of claim 12.

14. The apparatus of claim 13, further comprising:
a first sensor for sensing the first measurement data.

15. The apparatus of claim 14, wherein the first sensor comprises an antenna configured to receive the response resulting from an electromagnetic (EM) signal interacting with the subject's blood in a body part of the subject.

16. The apparatus of claim 13, further comprising:
at least one second sensor for sensing the second measurement data.

17. The apparatus of claim 16, wherein the at least one second sensor comprises a sensor selected from a group comprising: a skin surface temperature sensor; an ambient temperature sensor; a skin moisture level sensor; a humidity sensor; an accelerometer and an inertial measurement unit (IMU).

18. The apparatus of claim 13, further comprising:
a signal generator configured to generate the electromagnetic (EM) signal; and
an antenna in communication with the signal generator, the antenna configured to transmit the EM signal into the body part of the subject.

19. A wearable device configured to be worn on a body part of a subject, the wearable device comprising:
the apparatus of claim 13; and
an attachment mechanism configured to attach the wearable device to the body part of the subject such that the apparatus is maintained in contact with the subject's skin.

20. A system for determining a concentration of glucose in a subject's blood, the system comprising:
a processor configured to perform steps of the method of claim 1; and
a wearable device configured to be worn on the body part of the subject, the wearable device comprising:
a first sensor for sensing the first measurement data;
at least one second sensor for sensing the second measurement data; and
an attachment mechanism configured to attach the wearable device to the body part of the subject such that the wearable device is maintained in contact with the subject's skin.
